(19) United States
(12) Patent Application Publication
(10) Pub. No.: US 2021/0125695 A1
(43) Pub. Date: Apr. 29, 2021

(54) MEDICATION ADHERENCE MANAGEMENT

(71) Applicant: MEDISAFE PROJECT LTD., Nofit (IL)

(72) Inventors: Rotem SHOR, Nofit (IL); Oren SHAPIRA, Tel Aviv (IL); Ron ZEIDMAN, Kiryat Motzkin (IL); Amit WOLFENFELD, Karkur (IL); Anna PERSHUKOVA, Ramat Gan (IL)

(21) Appl. No.: 17/082,484

(22) Filed: Oct. 28, 2020

Related U.S. Application Data
(60) Provisional application No. 62/926,647, filed on Oct. 28, 2019.

(51) Int. Cl. G16H 20/10; G16H 40/67; G16H 10/60; G16H 50/20; G16H 50/70; G06Q 40/08; G06Q 10/10; A61J 7/04; G09B 19/00

(52) U.S. Cl. CPC G16H 20/10; G16H 40/67; G16H 10/60; G16H 50/20; G16H 50/70; A61J 2200/30; G06Q 10/109; A61J 7/0463; A61J 7/0418; A61J 7/0481; G09B 19/00; G06Q 40/08

(57) ABSTRACT

A method, system and product for medication adherence management. The method comprises obtaining data about a user, wherein the data comprises a prescription of a medication to be administered to the user. A user context data is calculated for the user. An intervention and timing thereof are determined for the user based on the user context. The determination may be performed using a prediction model that is trained with respect to a cohort, wherein the user is a member of the cohort. The intervention is implemented on the user at the determining timing of the intervention.

### CROSS-REFERENCE TO RELATED APPLICATION

This application claims the benefit of provisional patent application No. 62/926,647, entitled “A PLATFORM, DEVICE AND METHOD FOR MEDICATION ADHERENCE MANAGEMENT” filed Oct. 28, 2019, which is hereby incorporated by reference in its entirety without giving rise to disavowment.

### TECHNICAL FIELD

The present disclosure relates to medication management in general, and to personalized medication adherence management, in particular.

### BACKGROUND

Medication or drug adherence (sometimes also referred to as compliance) describes the degree to which a patient correctly follows medical instructions. Both the patient and the health-care provider affect adherence, and a positive physician-patient relationship is an important factor in improving adherence.

Worldwide, non-adherence is a major obstacle to the effective delivery of health care. Estimates from the World Health Organization (2003) indicate that only about 50% of patients with chronic diseases living in developed countries follow treatment recommendations. Adherence rates may be overestimated in the medical literature, as adherence is often high in the setting of a formal clinical trial but drops off in a “real-world” setting.

Major barriers to adherence are thought to include the complexity of modern medication regimens, poor “health literacy” and lack of comprehension of treatment benefits, the occurrence of undiscussed side effects, the cost of prescription medicine, and poor communication or lack of trust between the patient and her health-care provider. Efforts to improve adherence have been aimed at simplifying medication packaging, providing effective medication reminders, improving patient education, and limiting the number of medications prescribed simultaneously.

### BRIEF SUMMARY

One exemplary embodiment of the disclosed subject matter is a system comprising: a server; a plurality of mobile devices, each of which associated with a different user, wherein each mobile device retaining a mobile application that is configured to: obtain user-generated data about the associated user, wherein the user-generated data comprises a prescription of a medication to be administered to the associated user; obtain sensor information from the mobile device; communicate with said server over a network; and implement interventions determined by said server; wherein said server is configured to calculate a user context data of each user, wherein the user context data is determined based on retrieved user-generated data and sensor information transmitted by the mobile applications and based on enriched data from external data sources; wherein said server is configured to determine determining an intervention for each user based on the user context data that is aimed at improving adherence of each user to the user's prescription, wherein the intervention is performed using a prediction model that is trained with respect to a cohort, wherein the each user is a member of the cohort; wherein said server is configured to determine a timing of the intervention, wherein the timing of the intervention is determined based on the user context data; wherein said server is configured to instruct the mobile application to implement the intervention at the timing of the intervention.

Optionally, said system is further configured to determine a personalized processing time for each user, wherein the server is configured to determine the intervention and the timing of the intervention with respect to each user at the corresponding personalized processing time.

Optionally, the personalized processing time is determined based on a frequency of the prescription of each user.

Optionally, the processing time is determined based on a plurality of frequencies each of which corresponding to a different medication prescribed to the user.

Optionally, the processing time is determined based on a lowest common denominator of periods defined by the plurality of corresponding frequencies.

Another exemplary embodiment of the disclosed subject matter is a method comprising: obtaining data about a user, wherein the data comprises a prescription of a medication to be administered to the user; calculating a user context data for the user; determining an intervention for the user based on the user context data, wherein said determining the intervention is performed using a prediction model that is trained with respect to a cohort, wherein the user is a member of the cohort; determining a timing of the intervention, wherein the timing of the intervention is determined based on the user context data; implementing the intervention on the user at the timing of the intervention.

Optionally, said determining the intervention is performed at a processing time, wherein the method further comprises determining the processing time for the user, whereby the method provides personalized processing time for each user.

Optionally, the processing time is determined based on a frequency of the prescription of the medication.

Optionally, the data comprises a plurality of prescriptions of medications each of which having a corresponding frequency, wherein the processing time is determined based on the plurality of corresponding frequencies.

Optionally, said determining the processing time comprises determining a lowest common denominator of periods defined by the plurality of corresponding frequencies, wherein the processing time is determined based on the lowest common denominator.

Optionally, said implementing the intervention comprises verifying the intervention is applicable to the user; and in response to a successful verification of the intervention, providing the intervention to the user.

Optionally, the intervention is selected from a group consisting of at least one of: providing a reminder to apply the medication; coaching the user on how to administer the medication; providing positive reinforcement to the user; assisting user with obtaining the medication; providing a responsive action to an identification that the user internationally skips medication application; and providing a responsive action to an insurance coverage change.

Optionally, the method further comprises: tracking engagement of the user with the intervention; and utilizing the tracked engagement to improve the prediction model.

Optionally, said determining an intervention for the user comprises selecting the prediction model from a plurality of prediction models, wherein each model of the plurality of prediction models corresponds to a different cohort, wherein said selecting is performed to select the prediction model that is associated with the cohort of the user.

Optionally, said calculating the user context data comprises: obtaining user-generated data, and enriching the user-generated data with additional data, whereby determining a personalized user profile.

Yet another exemplary embodiment of the disclosed subject matter is a computer program product comprising non-transitory computer program instruction configured, when executed by a processor, to cause the processor to perform: obtaining data about a user, wherein the data comprises a prescription of a medication to be administered to the user; calculating a user context data for the user; determining an intervention for the user based on the user context data, wherein said determining the intervention is performed using a prediction model that is trained with respect to a cohort, wherein the user is a member of the cohort; determining a timing of the intervention, wherein the timing of the intervention is determined based on the user context data; implementing the intervention on the user at the timing of the intervention.

Optionally, said determining the intervention is performed at a processing time, wherein the method further comprises determining the processing time for the user, whereby the method provides personalized processing time for each user.

Optionally, the processing time is determined based on a frequency of the prescription of the medication.

Optionally, the data comprises a plurality of prescriptions of medications each of which having a corresponding frequency, wherein the processing time is determined based on the plurality of corresponding frequencies.

Optionally, said determining the processing time comprises determining a lowest common denominator of periods defined by the plurality of corresponding frequencies, wherein the processing time is determined based on the lowest common denominator.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows a flowchart diagram of a method, in accordance with the disclosed subject matter;

FIG. 2 shows a flowchart diagram of a method, in accordance with the disclosed subject matter;

FIG. 3 shows a flowchart diagram of a method, in accordance with the disclosed subject matter;

FIG. 4 shows a block diagram of an apparatus, in accordance with some exemplary embodiments of the disclosed subject matter; and

FIG. 5 shows a schematic illustration of an exemplary medication management system, in accordance with some exemplary embodiments of the disclosed subject matter; and

FIGS. 6A-6C shows flowchart diagrams of methods, in accordance with the disclosed subject matter.

### DETAILED DESCRIPTION

One technical problem dealt with by the disclosed subject matter is to assist medication takers in managing their medication regimen, such as to remind them to take medications on time, to take a correct dosage of medications, to monitor their adherence in taking the medications, or the like. Medication management may be desirable by patients as well as by clinicians. Many patients may face difficulties managing their medication regimen. Patients who take medication often miss doses, take wrong dosages, mix medications, confuse between medications, or the like. A change in a daily routine, the complexity in following the instructions, conditions on taking pills, or the like, may harden this task even more. In some cases, drug titration may also provide a challenge as the change of dosage is intended.

In some exemplary embodiments, a system that encourages patients to adhere and persistent in taking their medications, may be desired. However, adherence and persistence cannot be achieved through a system that provides a one solution to all patients. Different patients may have different needs and different behavior patterns. Even two patients that seem relatively similar, may act differently when facing the same condition and medication. As an example, one patient may proactively search for information, complete regular checkups, or the like; while another patient may only act on her health when absolutely necessary.

In some cases, in order to effectively change behavior, medication management platforms may need to take into account individual characteristics and to be implemented at precise timing. As an example, the same intervention to increase work ethic (highlighting norms about how people should behave) can be helpful for individuals high in self-control but counterproductive for impulsive individuals. Similarly, interventions to alleviate poverty (e.g., a campaign for signing up for health insurance) may be more effective when presented when people have spare cognitive capacity to consider them, such as after a harvest or a payday, versus when they are financially stressed.

One technical solution is a medication adherence management system that assists patients in managing their medication regimen by providing time specific adapted interventions, based on individual characteristics and health management behavior of the patient. In some exemplary embodiments, the system may be configured to leverage big data, Machine Learning (ML), deep learning, or the like, to understand the patient through their individual characteristics and through their health management activities, to provide the right intervention to the right patient at the right time. The individual characteristics may comprise demographic characteristics, characteristics related to medications, conditions, location, or the like. The health management activities may comprise medication adherence, health measurement tracking, consumption of educational content, or the like.

In some exemplary embodiments, the medication adherence management system may be configured to provide interventions to the patients to influence their behavior, such as by inducing the patient to take her pills, informing the patient about relevant risks, providing relevant information regarding diseases, suggesting certain activities to enhance the effectiveness of the medicine, or the like. In some exemplary embodiments, the intervention may be issued as a push notification via a mobile application associated with the medication adherence management system, an e-mail, a text message, a change in the application interface such as button location, a color, content cards, or the like.

In some exemplary embodiments, the medication adherence management system may monitor the user's behavior and determine the type of the intervention, the content of the intervention, the timing of issuing the intervention, or the like. The monitoring may be performed continuously, periodically, such as every hour, every predetermined time period, or the like. In some exemplary embodiments, as different subjects may relate to different parameters, a personalized period may be defined per user and utilized in the calculations related to her. The medication adherence management system may be configured to utilize machine learning models that are trained to provide the correct intervention based on the user behavior. The machine learning models may be updated and retrained, such a periodically, in response to certain events, such as when an adherence measurement decreases below a predetermined threshold, in response to a change in the data of the user, or the like.

In some cases, the personalized period may be defined based on the frequency as defined by the personal prescription of a drug, e.g., daily administration of the medication (e.g., 2 pills once a day), a weekly administration (e.g., 1 pill once a week), bi-weekly dosage (e.g., 2 pills every 2 weeks), or the like. In some cases, where a patient has more than one medication, the period may be determined based on the different prescriptions and their frequencies. Hence, a personalized timing to compute the desired intervention may be utilized for each patient, deferring computation with respect to some patients, without losing precision and accuracy with respect to other patients. In some exemplary embodiments, such a solution is efficient in computation resources for the application of the prediction model.

In some exemplary embodiments, the system may obtain as an input user-generated data. The system may be configured to transform and enrich the user-generated data, such as with demographic information, socio-economic status, treatment complexity information, or the like. In some exemplary embodiments, the data enrichment may allow to capture real-world phenomena and dependencies that influence user outcomes, such as socio-economic status, treatment complexity, comorbidities, patterns of application usage, adherence, or the like. In some exemplary embodiments, data related to the individual characteristics and the health management activities of the user may be collected by a mobile application, a body-worn device, or other platform of the system, such as by monitoring the user's interaction with different applications, by obtaining input from sensors of a mobile device of the user, or the like. Additionally or alternatively, the data may be explicitly added by the user to the application. As an example, the user may fill her characteristic data in the user profile in the application, such as gender, residential address, what medications the user takes and their prescribed frequencies, what are her health conditions, or the like. As another example, a schedule of the user may be determined based on a calendar application or other calendars in the user's social media profile. As yet another example, the user may mark her activities in the application via an adapted interface, such as by marking a dose as taken or skipped, reporting health measurements, answering survey, clicking a button, replying to a push notification or otherwise interaction with such notification, or the like. As yet another example, user activity may be tracked using body-worn sensors or other sensors used to track the user's activity, such as identifying when the user takes her pill.

In some exemplary embodiments, the medication adherence management system may be configured to analyze the individual characteristics and the health management activities of the user to determine adherence statistics of the user. The medication adherence management system may be configured to aggregate data collected on, or provided by, the user to calculate the adherence statics, such as how often the user takes her medications, how many medications the user missed, what time the user takes each medication, or the like.

In some exemplary embodiments, the medication adherence management system may be configured to enrich the data provided by the user or inferred based on data collected from the user or regarding the user, using data from third party sources, such as data from publicly available sources, data from commercial sources, or the like. As an example, the medication adherence management system may be configured to determine socio-economic data of the user based on the user's address, social activities, or the like. As another example, the medication adherence management system may be configured to determine most popular schedules and therapeutic area based on user medications. Based on the medications that the user takes, medication adherence management system may be configured to determine, using third party sources, what is the health condition of the user, how to schedule reminders to the user, personalize interventions to the user, or the like. As an example, in case the user adds reminders for ADVAIR™ to the platform, the medication adherence management system may utilize third party data sources to determine what condition is treated by ADVAIR™ and to conclude that the user suffers from asthma, even if the user has not added her condition to the platform. The medication adherence management system may be configured to send the user interventions personalized for users who suffer from asthma. As another example, in case the user adds GABAPENTIN™ to the platform, the medication adherence management system may be configured to determine, using third party data sources, that the most common schedules for taking GABAPENTIN™ is 2-3 times a day. Accordingly, the medication adherence management system may be configured to offer the user an easier way to schedule reminders with this frequency.

Additionally or alternatively, the medication adherence management system may be configured to determine additional data for the user using statistical models, machine learning models, deep learning models, or the like. Such models may be trained using previous data of the user and associated previously inferred data thereof. The previous data may comprise user generated data (e.g., data provided directly by the user or interaction of the user with the application), data calculated based on the user generated data, enriched data of the user generated data and data calculated based thereon, or the like. The previously inferred data may comprise user clustering based on medication and scheduling data, probability of user to churn, probability of user to miss specific dose, probability of user to click on push notification, expected number of days the user may be active, or the like.

In some exemplary embodiments, all the data of the user, e.g. user generated, calculated, enriched, and inferred data, may be combined into user context data. In some exemplary embodiments, the user context data may be a comprehensive user profile that is based on user-generated data, potentially enriches it with multiple internal and external sources, infers missing information, and places the user in relation to similar users, such as by assigning the user context data to a segment of users with similar characteristics. Each segment of users may comprise users sharing similar health conditions, taking similar medications, having similar social profile, having similar behavior, or the like. Different models may be applied for different user context segments, to determine interventions to be issued to the user.

In some exemplary embodiments, the medication adherence management system may be configured to assign an intervention to be issued to the user at the “right” time according to her user context data. In some exemplary embodiments, the “right” time for issuing the intervention to the user may be defined by the patient. Additionally or alternatively, the right time for issuing the intervention to the user may be automatically set by the medication adherence management system, such as a predetermine time before the appointed time of taking a medicine, in response to identifying certain events associated with the user (such as having a meal, performing a physical activity, or the like), in accordance with the user schedule, or the like.

In some exemplary embodiments, the medication adherence management system may be configured to apply machine learning models to determine an optimized intervention for each user context data status. The medication adherence management system may be configured to train such machine learning models based on the interaction of the user with the issued interventions, based on interventions issued for other users, based on the observed responses to the issued interventions, or the like. Additionally or alternatively, the machine learning models may be trained using training datasets generated and enriched by human experts.

In some exemplary embodiments, the medication adherence management system may be configured to actively initiate data collection by sending surveys to the user, such as on users' attitudes regarding their medication or treatment, reasons for non-adherence, or the like, and tracking user responses on provided interventions. Additionally or alternatively, the medication adherence management system may be configured to calculate similarity between different users and evaluate multiple outcome functions that can be related to retention, adherence, reaction to different interventions, or the like. This allows both to predict various outcomes for a single user and to infer the most likely reason for deviations from desired outcomes (e.g., non-adherence).

Additionally or alternatively, the medication adherence management system may be configured to perform intervention optimization based on the user responses on provided interventions. The best intervention, its timing, and its channel may be learned and optimized based on the user's engagement with the application, including the interventions themselves. The learning may be performed based on engagement of users in the same segment with the chosen interventions, user's previous engagement with similar interventions, or the like. The intervention optimization may comprise optimizing the intervention parameters such as the type of the intervention, parameters in the content of the intervention, the timing of the intervention, or the like.

In some exemplary embodiments, the disclosed subject matter may be utilized in a platform, system and method for social medication compliance.

In some exemplary embodiments, a platform may be provided for socially supporting medication adherence management. The platform may comprise a user application for managing medication compliance, data servers with memory thereon for storing compliance related data, and web servers running code to enable social medication adherence tracking.

In some exemplary embodiments, the medication compliance system may include computer code for enabling the personalization of system communications based on personal criteria, personal behavior tracking analytics, or the like.

In some exemplary embodiments, the medication compliance system may include personal user behavior tracking, optionally integrating medication administering gesture tracking based on a user movement, through one or more movement sensors. As an example, the sensors may be in or connected to wearable devices or connected devices, may be video cameras located in the environment in which the user is located, or the like.

In some exemplary embodiments, the medication compliance system may comprise predictive algorithms to enable enhanced alerting of users based on predictions of likely non-compliance.

According to some embodiments, a platform for social medication management is herein provided, which may include: a social media enabled website and medicine adherence services with complete social media network functionality; a mobile computing device enabled with a medication management application including a virtual pill box; a medication adherence database; and a group management portal for enabling user medication management data with a selected social group. In still further embodiments, the platform integrates connectivity to third party modules.

In some exemplary embodiments, the platform may comprise a data security module. In some exemplary embodiments, the platform may comprise a group alert module for alerting a selected social group of an event.

According to some embodiments, a method is provided for enabling social medication adherence management, comprising: downloading an application to a mobile device, where the application may be configured to display a virtual pill box; connecting to a medication adherence management platform; setting up a social group connected to a user via the platform, to help monitor user medication adherence; inserting medication data into the platform, via the application; inserting medication adherence data into the platform, via the application; and tracking medication adherence of the user, by the social group.

In some exemplary embodiments, the method may comprise synchronizing user medication data and user medication adherence data between the social group.

In some exemplary embodiments, the method may comprise utilizing one or more sensors. As an example, in wearable devices or other connected devices, to add user medication adherence data to the platform.

In some exemplary embodiments, the method may comprise tracking user movement to identify medication adherence data, and automatically entering such data to the platform.

In accordance with some embodiments, mobile applications (also referred to as “apps”) may be developed for various platforms or operating systems. Apps may optionally use the scanning capabilities of remote mobile or computing devices. The medication adherence system described herein, may comprise mobile application interfaces, desktop interfaces, Point Of Contact (POC) terminals, or the like. Such interfaces may enable compliance management from receipt of instructions from a practitioner, through the purchase or acquisition of medication, and during execution or usage of the medications. The system may be configured to enable continual or periodical monitoring of medication adherence, and sending of messages such as alerts and updates, to help encourage enhanced medication taking, and preventing forgetfulness or other factors active in decreasing compliance and adherence. The application or interface may include usage of graphics, video, voice, scanning, positioning data, user movement data and more to aid medication compliance monitoring. Applications in some cases may be customized for selected populations, conditions, environments, or the like.

In some exemplary embodiments, an application for mobile computing may comprise a virtual pill box, or a virtual medication box, representing the pills or the medications to be taken by a user during one or more time periods. The application may be utilized for medication adherence management. In some exemplary embodiments, the virtual pill box may enable the end user or the end user's social group to graphically view the pills or medications to be taken over or in a period of time, as well as the pills or medications that have been taken over or during a time period, the missed pills, or the like. In some exemplary embodiments, the virtual pills box may be presented in a graphical manner such as by displaying a display that is divided into multiple segments or compartments, reflecting the medications that a user should take and/or has taken in multiple periods of the day. Additionally or alternatively, the time of the pill or medication to be taken or that has been taken may be displayed. Optionally, the time of the pill or medication to be taken or that has been taken may be displayed in different fonts, colors, with other effects, or the like, to help the user or inform the user of medication taking adherence.

In some exemplary embodiments, a platform for social medication safety management may comprise a social media enabled website and medication adherence services with complete social media network functionality. The platform may further comprise a mobile computing device enabled with a medication management application. The medication management application may or may not include a virtual pill box. The platform may further comprise a group management portal, a medication adherence database and connectivity to third party modules.

In some exemplary embodiments, the system may be cloud-based and scalable to be used with any number of users. Further, in some embodiments, a distributed computing platform may be used, to help enable the analysis and processing of large amount of information accumulated substantially in real time, by using significant computing power to process data as necessary.

In some exemplary embodiments, the system may use external data from, for example, Health Maintenance Organizations (HMOs), medical organizations, government data sources, online sources, personal medical records, personal location data etc. Further, the system may make use of data from social networks, and may facilitate formation of user profiles and/or groups or group profiles, for example, to allow building of a social network related to a medication, condition etc. As an example, “objective” user data such as age, gender, race, etc. may be used to contribute to user profile setup, along with actual user behavior, user preferences, or the like.

In some exemplary embodiments, the system may analyze, optionally processing with artificial intelligence algorithms or other processing means, to create customized alerts, features, suggestions, predictions etc., to enhance the user experience. As an example, the system may enable analyzing personal usage patterns, interests, needs, limitation, or the like, to provide interfaces, services, suggestion, or the like to maximize user medication compliance and minimize the likelihood that a user will fail to take the medication.

In some exemplary embodiments, businesses or organizations may use user compliance tracking to enable matching or processing of such data in combination with data from drug companies, distributors, manufacturers, hospitals, HMOs etc. Such data processing may aid statistical evaluations of past, current and/or future medication usage, as well as predictions for usage, health threats, etc. immediate alerts medication use.

In some exemplary embodiments, data from the system may be used to deliver reports, process data to enable drug companies, distributors, manufacturers, hospitals, HMO's etc. to provide usage reports and predictions, or the like. Such data may help aid resource planning, production planning, health alert prediction etc.

In accordance with some embodiments, the system may integrate usage of wearable or other body tracking devices to help enable enhanced compliance. Accordingly, the system may be configured to connect to and correlate with a user's wearable device to help determine medication taking history and/or prediction of taking. For example, a user may make use of a wearable management sensor, optionally integrated into their hand watch or other devices, to use the device sensors to determine compliance data such as time of consumption. In some examples, the wearable device sensor may function as a gesture monitor, for example to determine if and when a user has made a medication opening/preparation movement, intake movement etc. In still further embodiments, specialized gestures related to medication compliance may be defined, monitored and tracked, to help determine user compliance optionally without relying on user data entry.

In accordance with additional embodiments, Predictive analysis may be used to predict user medication compliance and provide functionality enhancements in accordance. In some examples, user entered preferences, behavior tracking on various levels, or the like may be used to predict likely medication taking or failure to take medicines. In one example, compliance prediction may be based on analysis of user location, time, activity, and company used to predict likelihood of taking medication. As a result, if a certain situation is assumed to create a likelihood of forgetfulness to take medication, then a higher level of alert may be initiated to encourage the user to take medication. In some cases, other factors or combination of factors may be used. In a further example, based on system data analysis, it may be established that users in a certain geographical area are less likely to take medications in the evenings on weekends, in which case further measures such as group alerts may be used at these times to encourage extra vigilance in medication taking at these times for users in these places. Alerts or other smart events may be triggered by the system to compensate for increased likelihood of non-compliance.

The foregoing description of the embodiments of the disclosed subject matter has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the disclosed subject matter.

One technical effect of utilizing the disclosed subject matter is to create an easy-to-use, personalized technology that helps people better manage their medications, and offers healthcare companies meaningful insight into their daily behavior.

Another technical effect of utilizing the disclosed subject matter is to induce patients to take their medications in the right dose, on the right time, or the like, and dynamically determine additional future interventions to the patient based on the reaction of the patient with the current intervention.

Yet another technical effect of utilizing the disclosed subject matter is to allow both to predict various outcomes for a single user and to infer the most likely reason for deviations from desired outcomes (e.g., non-adherence).

Yet another technical effect of utilizing the disclosed subject matter is to improve resource utilization of the system, so as to allocate computational resources in a heterogeneous manner, and utilize such resources based on the parameters of the different users. In some cases, some users may require frequent re-consideration in view of their activity, and computational resources may be allocated to handle them accordingly, while other users may require less frequent reconsideration and the time that passes between prediction computations for those users may be greater, sparing computational and memory resources from the platform.

The disclosed subject matter may provide for one or more technical improvements over any pre-existing technique and any technique that has previously become routine or conventional in the art. Additional technical problem, solution and effects may be apparent to a person of ordinary skill in the art in view of the present disclosure.

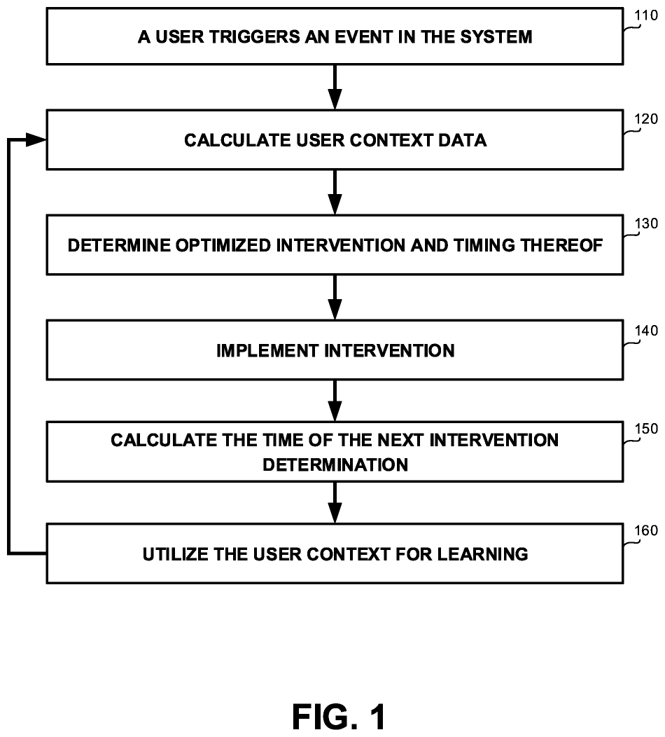

Referring now to FIG. 1, showing a flowchart diagram of a method, in accordance with the disclosed subject matter.

On Step 110, a user may trigger an event in the system. In some exemplary embodiments, the event may comprise one or more time related actions of the user. As an example, the event may be creating a profile in the system, updating a profile in the system, answering a survey, interacting with the application, taking a dose (e.g., marking a dose as taken), missing a dose (e.g., marking a dose as skipped), or the like. As another example, the event may indicate the user not performing an action, such as the user not marking a pill as taken in a due time. Additionally or alternatively, the event may be automatically determined based on sensors of a device associated with the user, such as movement sensors, location sensors, thermometers, or the like, based on data obtained from social networking platforms, or the like. As an example, the event may be indicating an extreme change in the temperature, a barometric pressure sensor or using location sensors to determine the user is away from a location in which the medication is located, or the like. As another example, the event may be having a meal, performing a physical activity, measuring a high blood pressure, or the like.

On Step 120, the user context data may be calculated. In some exemplary embodiments, each user may be associates with a profile in the system. The profile may comprise personal and demographic data of the user, such as gender, age, zip code, medications the user is supposed to take, the supposed timing of taking each medication, health conditions, schedules, or the like.

In some exemplary embodiments, the profile may comprise user generated data. The user generated data may be data explicitly added by the user to the application or recorded from user interactions with the application. Additionally or alternatively, the profile may comprise user calculated data. The user calculated data may comprise transformations or aggregations of user-generated data, such as adherence statistics, or the like. Additionally or alternatively, the profile may comprise user enriched data. The user enriched data may be data derived from combining user-generated data and data from third party sources, such as commercial data, socio-economic data based on user location, most popular schedules and therapeutic area based on user medication, or the like. Additionally or alternatively, the profile may comprise user inferred data. The user inferred data may be a data based on outputs from various statistical, machine learning, and deep learning models trained on user generated, calculated, enriched, and previously inferred data. As an example, the user inferred data may comprise user cluster based on medication and scheduling data, probability of user to churn, probability of user to miss specific dose, probability of user to click at push notification, expected number of days the user will be active within the next 7 days, or the like. Additionally or alternatively, the profile may comprise user context data which combines user generated, calculated, enriched, and inferred data.

In some exemplary embodiments, the context data of the user may be calculated or updated based on the event of Step 110. Updating the user context data may comprise updating at least one of user generated, calculated, enriched, and inferred data, based on the triggered event.

In some exemplary embodiments, the user context data may be retrieved from a data repository and potentially updated in view of data collected since the last update of the user context data.

On Step 130, an optimized intervention and the timing for implementing thereof may be determined.

In some exemplary embodiments, the intervention may be a message or a change in the application interface (such as a change in button location) designed to influence the user's behavior. Additionally or alternatively, the intervention may be a reminder, a notification in the mobile device of the user, or the like, that may be implemented to actively remind the user of a desired activity.

Additionally or alternatively, the intervention may be designed to gather additional data about the user to determine a better intervention, to understand the user's context, or the like. As an example, the intervention may comprise a request to the user to update her profile, to mark a pill as taken or missed, or the like.

Additionally or alternatively, the intervention may comprise providing content, such as articles, or any other similar content that is related to the event. As an example, the intervention may comprise providing information about side effects of the pill. As another example, the intervention may comprise a suggestion for a replacement medication, dietary supplements to be taken with the medicine, or the like.

Additionally or alternatively, the intervention may be based on activity patterns of the user, such as may be observed over time. As an example, the user may be skipping every third administration of her drug. Such a pattern may be indicative of an intentional desire to reduce medication costs. Appropriate intervention may be then determined, such as suggesting replacement medication at a lower budget, suggesting to apply to financial assistance programs, or the like.

In some exemplary embodiments, an optimized intervention may be assigned for the user according to the user context data of the user. In some exemplary embodiments, the optimized intervention may be determined using machine learning models. In some exemplary embodiments, the user context data may be utilized to determine a cohort relevant to the user, such as users exhibiting similar behavior and having similar properties. In some exemplary embodiments, each cohort may be associated with a different prediction model that is used to determine the optimized intervention. In some exemplary embodiments, once a model is selected, the optimized intervention may be determined as the intervention with the highest score according to the selected prediction model.

In some exemplary embodiments, an optimized time for issuing the optimized intervention may be determined according to the user context data. The optimized time may be determined based on the type of the optimized intervention, habits of the user, the urgency of the event, or the like. As an example, some interventions may be configured to be sent immediately to the user. As another example, some interventions may be scheduled to be sent a predetermined time before the supposed timing of taking a pill, a predetermined time after the supposed timing of taking a pill, or the like. Additionally or alternatively, some interventions may be scheduled to specific contexts, such as when the user is home resting, when the user is about to go to sleep, when the user arrives to her office, or the like.

On Step 140, the intervention may be implemented. In some exemplary embodiments, the intervention may be implemented. The intervention may be implemented by presenting the intervention to the user via a channel, such as by a push notification, an email, a text message, in-app content cards, or the like. In some exemplary embodiments, the intervention may be implemented by providing a synthesized human voice audio that is provided via a phone call. Additionally or alternatively, the intervention may be determined to be a human-intervention, where a human different than the user is notified and is tasked with assisting the user. For example, the human intervention may be implemented by texting or otherwise notifying the user's care taker that the user had failed to take her medication. As another example, the human intervention may be implemented by a means of an email that is sent to the user's parents or guardians informing them of the fact that the user is hesitant to apply the medication that was prescribed to her, so as to encourage the guardians to assist her in overcoming the challenge she is facing.

On Step 150, the time of the next intervention may be calculated. In some exemplary embodiments, the optimized intervention may be determined as an intervention that should be repeated, such as when a similar trigger occurs, periodically, after a predetermined timeframe, or the like. The time for issuing the next similar intervention may be calculated based on the user context data, based on the type of the intervention, based on the effectiveness of the intervention as observed on the user, or the like. Calculating the time of the next intervention may reduce time between obtaining a similar trigger and issuing the intervention, which may comprise recalculating and sending a new user context data, applying machine learning models to determine the intervention, or the like.

On Step 160, the user context may be utilized for additional learning.

In some exemplary embodiments, the optimized intervention, its timing, and the channel utilized to issue it, may be learned and optimized based on the user's engagement with the application, such as the user engagement with the interventions themselves. Machine learning models that are trained using the user context data and the optimized intervention may be applied to fine-tune the intervention parameters. In some exemplary embodiments, a segment level indicating the engagement of users in the same segment (e.g., segment of users with similar characteristics) with the chosen interventions may be determined. Additionally or alternatively, an individual user level indicating the user's previous engagement with similar interventions, may be determined. The machine learning models may be re-trained using the segment level and the individual user level. The re-training may aim to fine-tune the intervention parameters such that the selected intervention achieves peak performance (e.g., adherence, persistence, or the like) while minimizing alert fatigue.

It may be noted that different machine learning models may be utilized for different user segments, for different instance, or the like.

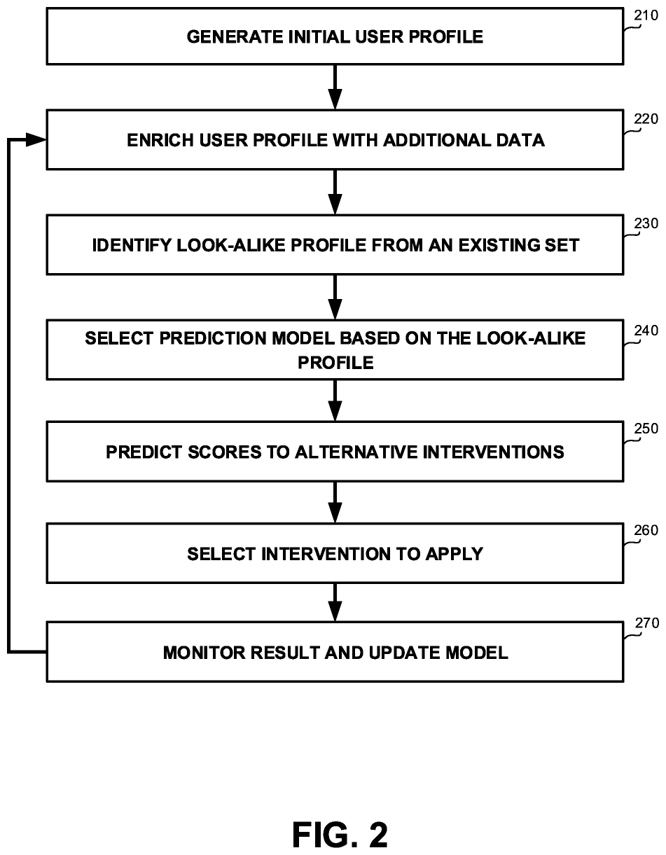

Referring now to FIG. 2, showing a flowchart diagram of a method, in accordance with the disclosed subject matter.

On Step 210, an initial user profile may be generated. The user profile may be generated based on user-provided data, based on automatically obtained data, or the like. In some exemplary embodiments, the user profile may comprise the user context data.

On Step 220, the user profile may be enriched with additional data. The additional data may be obtained by tracking activity of the user, by querying external data sources, by deducing information from previously gathered data, or the like. The updated user profile may be retained for future usage to avoid a requirement to re-calculate the user profile from scratch. Instead, the previously retained user profile may be retrieved from a data storage and updated according to additional data that has become available.

In some exemplary embodiments, user profile may be enriched using data obtained from third party sources. In some exemplary embodiments, Customer Relationship Management (CRM) of medical professionals, such as doctors, nurses, patient support services, or the like, may be utilized to enrich the profile with information not provided by the user or to correct incorrect information. For example, the user may indicate her weight. However, if the CRM indicates an up-to-date measurement of the user's weight, the up-to-date measurement may be retrieved and used. As another example, the CRM may include prescriptions prescribed to the user. Additionally or alternatively, a pharmacy management system may be utilized to enrich the user profile. For example, the issued drugs may be indicated. In some cases, the actual issued drug may be different than the prescribed drug, e.g., being a generic version thereof, being an alternative drug thereto, or the like. As another example, the pharmacy management system may indicate that the user purposefully purchased a reduced amount of medications in comparison to the user's prescription. Additionally or alternatively, a system retaining Electronic Health Records (EHR) or Electronic Medical Records (EMR) may be utilized to augment the user profile with health-related information. Additionally or alternatively, medical insurance platforms may be utilized to enhance to insurance-related data regarding the user, such as indicating the user's current or past coverage.

On Step 230, a look-alike profile may be selected for the user. The look-alike profile may be selected from a pre-existing set of profiles based on similarity measurements from the user profile. For example, a similarity function may be utilized to compute a similarity degree between the user profile and each profile in the pre-existing set, so as to identify the profile that is most similar to that of the user. In some exemplary embodiments, each look-alike profile may represent a cluster of profiles that were previously encountered by the platform and that were deduced to behave in a similar manner. In some exemplary embodiments, the look-alike profile may be a representative profile of a cohort.

On Step 240, a prediction model may be selected based on the selected look-alike profile. In some exemplary embodiments, each look-alike profile may be associated with a prediction model that was trained to provide predictions for the represented cohort. In some exemplary embodiments, different look-alike profiles may be associated with different prediction model types (e.g., using different machine learning algorithms, deep learning algorithms, or the like). Additionally or alternatively, each prediction model may be trained using different training data that was obtained from the relevant cohort.

On Step 250, using the selected prediction model, a score for each alternative potential intervention may be computed. The score may be indicative of whether the intervention will positively affect user in the cohort. For example, a relatively high score may indicate that the intervention may increase the likelihood that the user will correctly administer her medicine, timely take her drug, adhere to the prescription she received, or the like.

On Step 260, an intervention may be selected to be applied for the user. In some exemplary embodiments, the selection may be based on the predicted scores of Step 250. It is noted that in some cases, the top-scored intervention may be selected. Additionally or alternatively, the N top-scored interventions may be identified and a random selection may be made between them to allow the platform to be exposed to different interventions and measure their effectiveness.

On Step 270, the results of the intervention may be monitored. Additionally or alternatively, the model may be updated accordingly. In some exemplary embodiments, the results may be added to a re-training dataset that is used to improve the prediction model over time. In some exemplary embodiments, the results of the intervention may comprise whether the user engaged with the intervention, at what time, and in what manner. Additionally or alternatively, the results of the intervention may comprise whether the user adhered to her prescription in the next medication administration time or at a future timeframe (e.g., following day, following week, or the like).

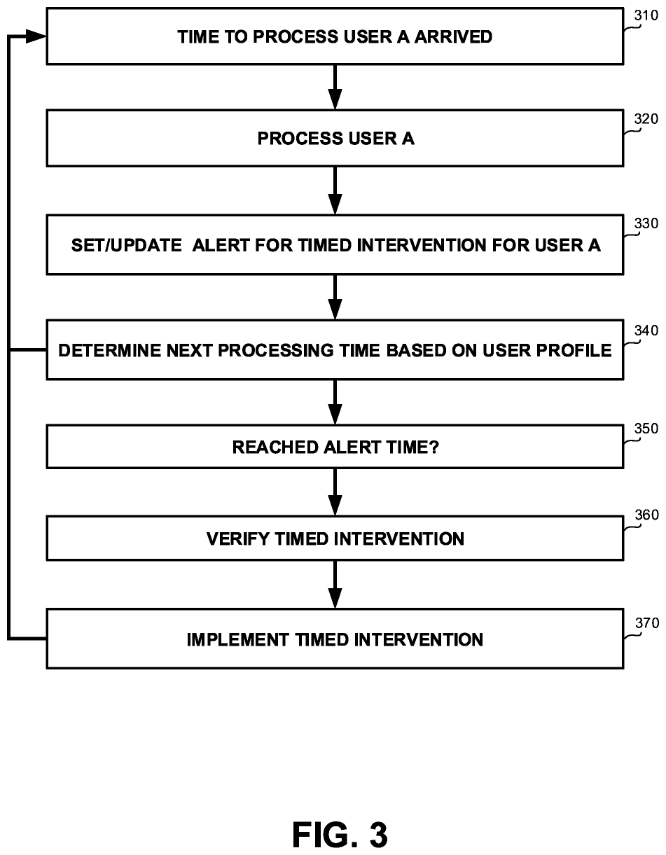

Referring now to FIG. 3, showing a flowchart diagram of a method, in accordance with the disclosed subject matter. FIG. 3 illustrates analysis of a single user, referred to as User A. It should be understood that each user may be processed in a similar manner, thereby achieving different results for different users.

On Step 310, the time to process User A arrived. In some exemplary embodiments, the time to process User A may have been determined previously (e.g., On Step 340 of a previous processing iteration). Additionally, or alternatively, the time to process User A may be determined based on an action of the user (e.g., the user registering to the platform), based on a detection of an event (e.g., the user missing her medication; the user reaching a predetermined location; the user's context changing to a predetermined context; or the like), or the like.

On Step 320, User A may be processed. The processing may be performed in accordance with the methods depicted in FIGS. 1-2, or the like. The processing may be based on the user context data of User A. Additionally, or alternatively, the processing may result in a determination of an intervention, such as by selecting an intervention from an alternative set of interventions using a prediction model. Additionally, or alternatively, the processing may determine a time and context for triggering the intervention.

On Step 330, in case the intervention determined on Step 320 is not an immediate intervention, an alert may be set for a determined timed intervention for User A. In some exemplary embodiments, if a previously determined timed intervention is updated, such as by modifying the intervention type and/or the intervention time, a previously set alarm may be updated.

Once the alert time is reached (Step 350), the timed intervention may be verified (Step 360) and implemented (Step 370). In some exemplary embodiments, it may be verified that the time intervention is still relevant before the timed intervention is implemented. For example, the intervention may be related to providing a reminder to take a medication. However, if the user has already indicated, e.g. by checking a checkbox in the App, that the medication was taken, no reminder is required. As yet another example, if the intervention is set to be implemented once User A is at her home, before implementing the intervention, it may be verified that the user is indeed at her home as was a-priori predicted. If the verification process fails, Step 330 may be re-performed to update/remove the timed intervention.

On Step 340, a next processing time for User A may be determined. In some exemplary embodiments, the next processing time may be determined and set before the time of the timed intervention, allowing for an update of the timed intervention in view of newly accumulated data. Additionally, or alternatively, the next processing time may be determined to be at the same time or within a short timeframe of the timed intervention, so as to allow to process user A and determine a new intervention (e.g., by setting a next processing time to be addressed in Step 310). Additionally, or alternatively, in the processing no timed intervention may be determined, and the next processing time may be a time in which a new intervention may be determined.

In some exemplary embodiments, the next processing time may be determined based on the user profile of User A. In some exemplary embodiments, the next processing time may be determined based on a frequency of a prescription of User A. For example, in case User A has a frequency of taking a pill once a week, the next processing time may be determined in proximity (e.g., a predetermined time before) to the next scheduled time to take User A's next pill. In some exemplary embodiments, User A may have several prescriptions at different frequencies. The determined time may be based on several frequencies, such as taking into account the closest next administration time of a medication according to any prescription. In some exemplary embodiments, the next processing time may be determined based on a determined period. The period may be the same period as the highest frequency medication. Additionally, or alternatively, the lowest common denominator of all periods of the different prescriptions may be determined and used as to determine the period to be used for User A. For example, consider User A having a medication to be taken once every four days and another taken once every six days. The lowest common denominator of two days may be determined and used, so that User A's interventions are looked upon every two days. In another example, there may be a daily medication and a weekly medication, resulting in a lowest denominator of a single day. So, User A's interventions may be re-processed on a daily basis. As can be appreciated from the above, the disclosed subject matter may provide a platform with user-personalized and heterogeneous processing periods to different users.

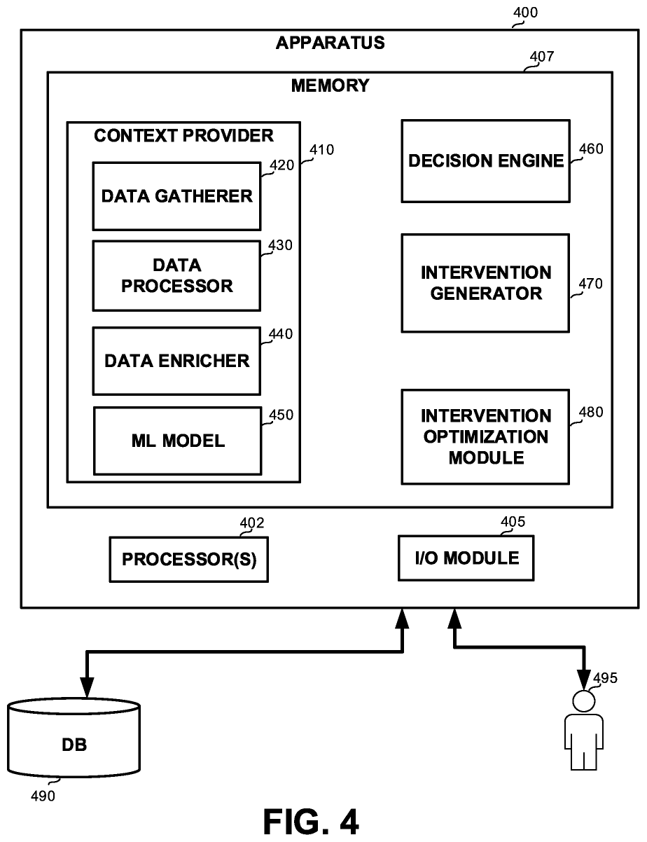

Referring now to FIG. 4 showing a block diagram of an apparatus, in accordance with some exemplary embodiments of the disclosed subject matter. An Apparatus 400 may be configured to support parallel user interaction with a real world physical system and a digital representation thereof, in accordance with the disclosed subject matter.

In some exemplary embodiments, Apparatus 400 may comprise one or more Processor(s) 402. Processor 402 may be a Central Processing Unit (CPU), a microprocessor, an electronic circuit, an Integrated Circuit (IC) or the like. Processor 402 may be utilized to perform computations required by Apparatus 400 or any of it subcomponents.

In some exemplary embodiments of the disclosed subject matter, Apparatus 400 may comprise an Input/Output (I/O) module 405. I/O Module 405 may be utilized to receive input from a user, such as, for example receiving indication of taking or missing a medicine, obtaining medical information, or the like. Additionally or alternatively, I/O Module 405 may be utilized to provide an output to a user, such as providing an intervention to the user.

In some exemplary embodiments, Apparatus 400 may comprise Memory 407. Memory 407 may be a hard disk drive, a Flash disk, a Random Access Memory (RAM), a memory chip, or the like. In some exemplary embodiments, Memory 407 may retain program code operative to cause Processor 402 to perform acts associated with any of the subcomponents of Apparatus 400.

In some exemplary embodiments, Context Provider 410 may be configured to generate a user context profile of User 495. Context Provider 410 may be configured to collect and enrich the user's raw data with insights gathered from various sources.

In some exemplary embodiments, a Data Gatherer 420 may be configured to collect data about User 495, such as input user-generated data provided by User 495, data collected from databases, or the like. A Data Processor 430 may be configured to calculate and aggregate information from the data gathered by Data Gatherer 420. Data Enricher 440 may be configured to enrich the data with known information inferred from user generated data.

In some exemplary embodiments, Context Provider 410 may be configured to utilize a Machine Learning (ML) Model 450 to infer additional data related to User 495, such as treatment complexity, comorbidities, patterns of application usage, adherence, or the like.

In some exemplary embodiments, a Decision Engine 460 may be configured to assign interventions to a user at the right time according to user context data provided by Context Provider 410. Decision Engine 460 may be configured to determine the intervention based on similarity between user context data of different users. Decision Engine 460 may be configured to evaluate multiple outcome functions that can be related to retention, adherence, reaction to different interventions, or the like. Decision Engine 460 may be configured to determine the timing of issuing each intervention.

In some exemplary embodiments, Intervention Generator 470 may be configured to generate interventions and implement them by providing the interventions to users based on decisions of a Decision Engine 460.

In some exemplary embodiments, Intervention Optimization Module 480 may be configured to optimize the performance of Decision Engine 460 based on the reaction of the user to the intervention, engagement with the intervention, feedback from the user, monitored activity of the user after the intervention, or the like. Intervention Optimization Module 480 may be configured to analyze the user's engagement with the intervention. Intervention Optimization Module 480 may be configured to apply machine learning models to fine-tune the intervention parameters such that the selected intervention achieves peak performance (adherence, persistence, etc.) while minimizing alert fatigue.

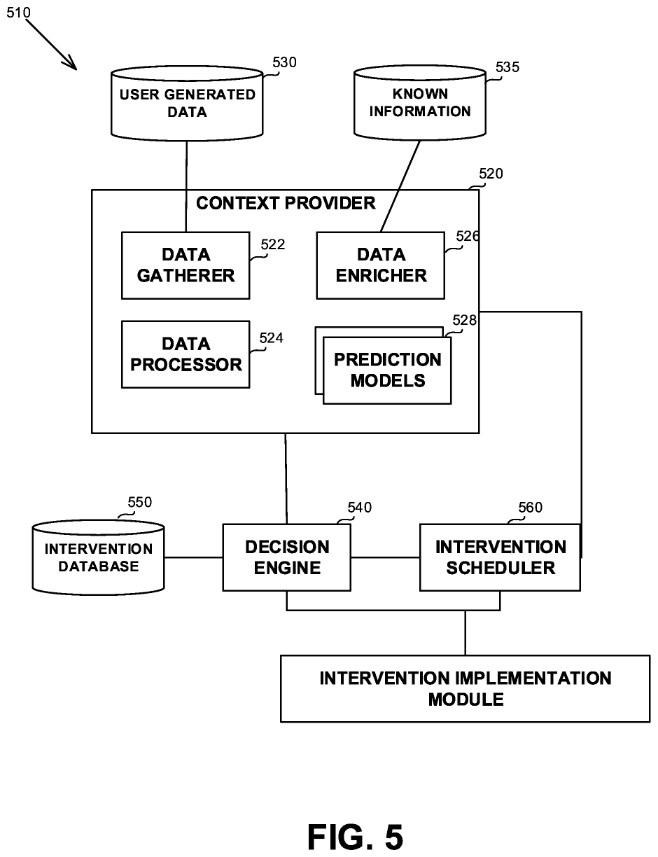

Referring now to FIG. 5 showing a schematic illustration of an exemplary medication management system, in accordance with some exemplary embodiments of the disclosed subject matter.

In some exemplary embodiments, a System 510 may be a medication management system utilized to assist a user in managing her medication regimen, improving adherence, or the like.

In some exemplary embodiments, System 510 may comprise a Context Provider 520. Context Provider 520 may be configured to generate User Context Data for the user. Context Provider 520 may be configured to collect and enrich raw data of the user with insights gathered from various sources.

In some exemplary embodiments, Context Provider 520 may be configured to utilize a Data Gatherer 522 to collect user generated data. Data Gatherer 522 may be configured to obtain data about the user, such as from a System Database 530. System Database 530 may comprise data provided by the user, such as by interacting with System 510, an application thereof, or the like.

In some exemplary embodiments, Context Provider 520 may be configured to utilize a Data Processor 524 to calculate and aggregate information from the data obtained by Data Gatherer 522.

In some exemplary embodiments, Context Provider 520 may be configured to utilize a Data Enricher 526. Data Enricher 526 may be configured to enrich the user calculated data generated by Data Processor 524. Data Enricher 526 may be configured to infer additional information about the user, such as socio-economic, schedule, or the like. In some exemplary embodiments, Data Enricher 526 may be configured to utilize data from other databases to obtain additional information, such as from a Database 535.

In some exemplary embodiments, Context Provider 520 may be configured to utilize Machine Learning models 528 to add data insights to the user enriched data determined by Data Enricher 526. As an example, the user inferred data may comprise segmentations of the user data, predictions based on user actions in the application, or the like. In some exemplary embodiments, Machine Learning models 528 may be trained to determine the effectiveness of the interventions on the user. As an example, Machine Learning models 528 may be trained to predict if the user is going to become non-adherent in a future predetermined time frame (such as next week, next month, or the like) if no interventions are issued thereto, if partial intervention are issued thereto, or the like.

In some exemplary embodiments, System 510 may comprise a Decision Engine 540. Decision Engine 540 may be configured to assign interventions to the user at the right time according to her user context data inferred by Context Provider 520. Decision Engine 540 may be configured to select an intervention from an Intervention Database 550. Decision Engine 540 may be configured to utilize machine learning models for selecting the appropriate intervention, timing of issuing the intervention, or the like.

In some exemplary embodiments, in response to the user triggering an event in System 510, such as creating a profile, marking taking or missing a dose, or the like, Context Provider 520 may recalculate the user's entire context by utilizing Data Gatherer 522, Data Processor 524, Data Enricher 526 and Machine Learning models 528. The recalculated context may be sent to the Decision Engine 540, so it can calculate which interventions should be sent to the user and when.

In some exemplary embodiments, System 510 may comprise an Interventions Scheduler 560. Intervention Scheduler 560 may be configured to determine the timing for issuing the current intervention and the next interventions. Interventions that Decision Engine 540 decides that should be sent immediately are sent to the user. Intervention Scheduler 560 may be configured to calculate the time of the next future intervention from the same type that should be sent to the user, without waiting to an additional trigger from the user. Intervention Scheduler 560 may utilize data from Context Provider 520 to calculate the time of the next future intervention. Additionally, or alternatively, on the time that the next intervention is scheduled, Context Provider 520 may be configured to recalculate the user context data and resend it to Decision Engine 540 before issuing the specific intervention to the user.

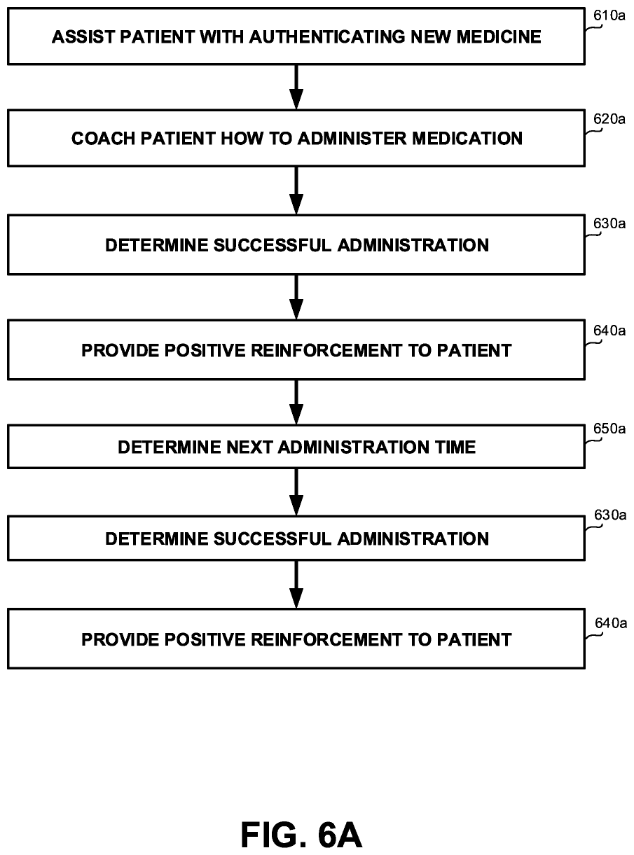

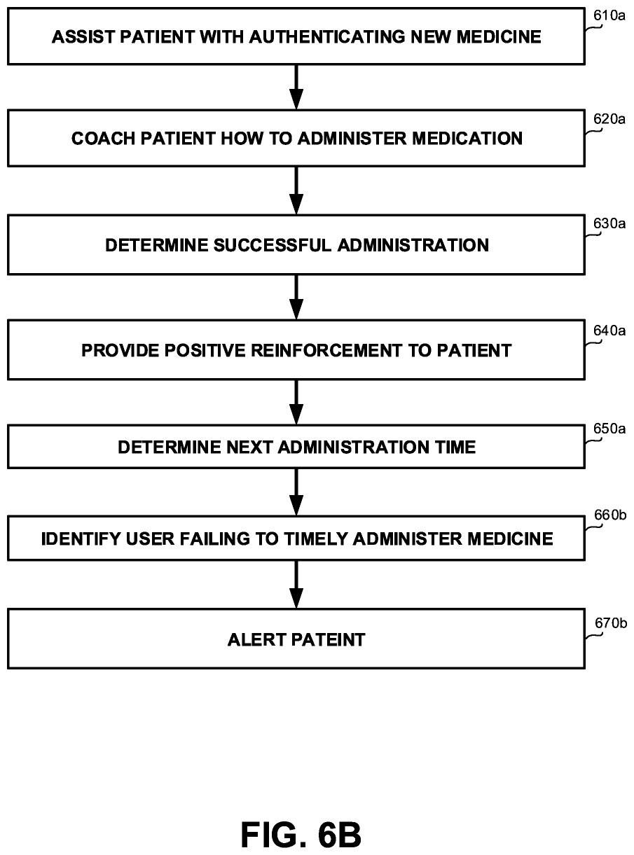

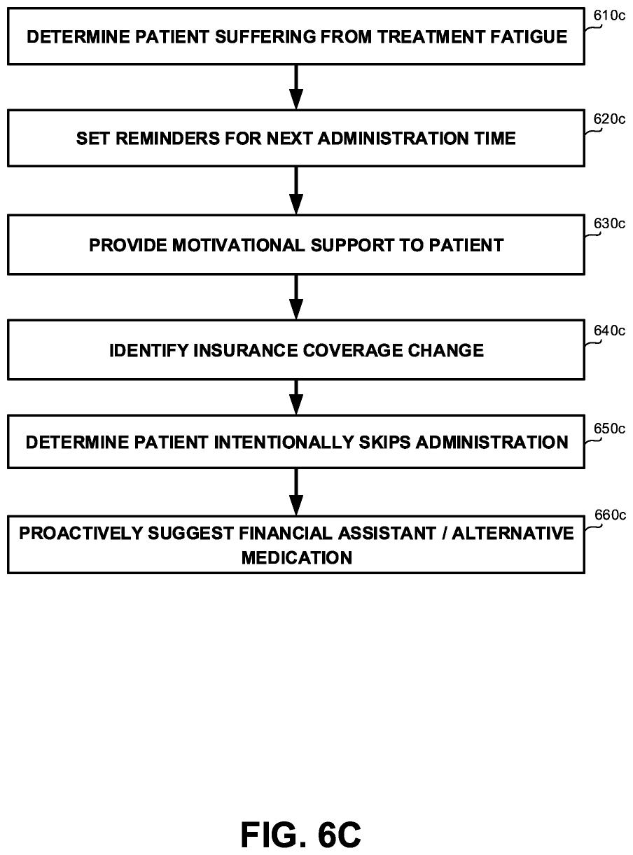

Referring now to FIGS. 6A-6C showing flowchart diagrams of methods of heterogeneous patient joineries, in accordance with the disclosed subject matter.

In FIG. 6A, a journey of a patient who is newly diagnosed with a chronic condition is illustrated. The platform may determine the characteristics of the patient and determine that the patient is new to treatment and to specialty medicine.

Accordingly, the first intervention of the platform may be aimed at assisting the patient with the prior authenticating process with relation to the new medicine, potentially including tracking shipment of the new medicine to her home (610a). Once the medication is available, the platform may provide another intervention that is aimed to coach patient on how to administer the medication (620a). Consider, for example, an injection. The patient may be scared and reluctant to administer the injection for the first time. The intervention may include showing a media clip on how to administer the injection; providing textual explanations, tutorials on how to prepare and take the first injection, or the like. After the platform determined that the patient successfully administered the medicine (630a), an intervention in the manner of positive feedback to reinforce the accomplishment of the patient may be provided. Such an intervention may be aimed at keeping the patient focused on the course of the treatment. A next administration time may be determined (650a) and if at that time, a successful administrator is identified once again (630a), an additional positive reinforcement may be provided.

As can be appreciated from this patient journey, no reminder is required as the patient is considered to be well aware of her time to take the medicine and her challenge is related to unfamiliarity with the new medicine. It is further noted that avoiding unnecessary reminders may reduce alarm fatigue for the patient

In FIG. 6B, the journey of the same patient is shown. However, in FIG. 6B. the patient failed to timely administer the medicine (660b). In view of the such event, the patient is identified to be related to a cohort of forgetful patients, and alerts may be set and provided for the patient (670b).

In FIG. 6C, a journey of a different patient is shown—a patient who is experienced in the treatment. The patient may be managing multiple conditions and therefore has several medications to manage. As a result, the patient may be experiencing treatment fatigue. After the platform determines the patient is suffering from treatment fatigue (610c), such as based on tracked information, based on the suer context data, or the like, the platform may support the patient in a manner that is useful to cope with such fatigue. Reminders of next administration time may be set (620c). The platform may assist the patient in staying engaged with her treatment needs. Additionally, or alternatively, the platform may provide motivational support by helping the patient track her health measurements so she can visualize the effects of her treatment (630c). In some exemplary embodiments, an insurance coverage change may be identified (640c), and it may be determined that the patient intentionally skips administration (650c). The platform may determine that the patient is now concerned about affording her specialty medication and attempted to spread out the treatment by skipping a dose here or there. The platform may intervene to find out her reason for skipping and proactively engages her in the financial assistance programs (660c).

It is noted that a patient intentionally skipping administration may be differentiated from a patient forgetting to take the medication using different features and properties. For example, if the patient receives the alert when she is at home alone, if she skips the administration, this may be indicative that the skip is intentional, in contrast to an unintentional administration if the alarm is triggered when the patient is unable to administer the medicine because she is away from home or entertaining guests.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The disclosed subject matter is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

What is claimed is:

1. A system comprising:
a server;
a plurality of mobile devices, each of which associated with a different user, wherein each mobile device retaining a mobile application that is configured to:
obtain user-generated data about the associated user;
obtain sensor information from the mobile device;
communicate with said server over a network; and
implement interventions determined by said server;
wherein said server is configured to calculate a user context data of each user, wherein the user context data is determined based on retrieved user-generated data and sensor information transmitted by the mobile applications and based on enriched data from external data sources, wherein the user context data comprises a prescription of a medication to be administered to the associated user;
wherein said server is configured to determine determining an intervention for each user based on the user context data that is aimed at improving adherence of each user to the user's prescription, wherein the intervention is performed using a prediction model that is trained with respect to a cohort, wherein the each user is a member of the cohort;
wherein said server is configured to determine a timing of the intervention, wherein the timing of the intervention is determined based on the user context data;
wherein said server is configured to instruct the mobile application to implement the intervention at the timing of the intervention.

2. The system of claim 1, wherein said system is further configured to determine a personalized processing time for each user, wherein the server is configured to determine the intervention and the timing of the intervention with respect to each user at the corresponding personalized processing time.

3. The system of claim 2, wherein the personalized processing time is determined based on a frequency of the prescription of each user.

4. The system of claim 2, wherein the processing time is determined based on a plurality of frequencies each of which corresponding to a different medication prescribed to the user.

5. The system of claim 2, wherein the processing time is determined based on a lowest common denominator of periods defined by the plurality of corresponding frequencies.

6. A method comprising:
obtaining data about a user, wherein the data comprises a prescription of a medication to be administered to the user;
calculating a user context data for the user based on the data about the user;
determining an intervention for the user based on the user context data, wherein said determining the intervention is performed using a prediction model that is trained with respect to a cohort, wherein the user is a member of the cohort;
determining a timing of the intervention, wherein the timing of the intervention is determined based on the user context data;
implementing the intervention on the user at the timing of the intervention.

7. The method of claim 6, wherein said determining the intervention is performed at a processing time, wherein the method further comprises determining the processing time for the user, whereby the method provides personalized processing time for each user.

8. The method of claim 7, wherein the processing time is determined based on a frequency of the prescription of the medication.

9. The method of claim 7, wherein the data comprises a plurality of prescriptions of medications each of which having a corresponding frequency, wherein the processing time is determined based on the plurality of corresponding frequencies.

10. The method of claim 7, wherein said determining the processing time comprises determining a lowest common denominator of periods defined by the plurality of corresponding frequencies, wherein the processing time is determined based on the lowest common denominator.

11. The method of claim 6, wherein said implementing the intervention comprises verifying the intervention is applicable to the user; and in response to a successful verification of the intervention, providing the intervention to the user.

12. The method of claim 6, wherein the intervention is selected from a group consisting of at least one of:
providing a reminder to apply the medication;
coaching the user on how to administer the medication;
providing positive reinforcement to the user;
assisting user with obtaining the medication;
providing a responsive action to an identification that the user internationally skips medication application; and
providing a responsive action to an insurance coverage change.

13. The method of claim 6 further comprises:
tracking engagement of the user with the intervention; and
utilizing the tracked engagement to improve the prediction model.

14. The method of claim 6, wherein said determining an intervention for the user comprises selecting the prediction model from a plurality of prediction models, wherein each model of the plurality of prediction models corresponds to a different cohort, wherein said selecting is performed to select the prediction model that is associated with the cohort of the user.

15. The method of claim 6, wherein said calculating the user context data comprises:
obtaining user-generated data, and enriching the user-generated data with additional data, whereby determining a personalized user profile.

16. A computer program product comprising non-transitory computer program instruction configured, when executed by a processor, to cause the processor to perform:
obtaining data about a user, wherein the data comprises a prescription of a medication to be administered to the user;
calculating a user context data for the user based on the data about the user;
determining an intervention for the user based on the user context data, wherein said determining the intervention is performed using a prediction model that is trained with respect to a cohort, wherein the user is a member of the cohort;
determining a timing of the intervention, wherein the timing of the intervention is determined based on the user context data;
implementing the intervention on the user at the timing of the intervention.

17. The computer program product of claim 16, wherein said determining the intervention is performed at a processing time, wherein the method further comprises determining the processing time for the user, whereby the method provides personalized processing time for each user.

18. The computer program product of claim 17, wherein the processing time is determined based on a frequency of the prescription of the medication.

19. The computer program product of claim 17, wherein the data comprises a plurality of prescriptions of medications each of which having a corresponding frequency, wherein the processing time is determined based on the plurality of corresponding frequencies.

20. The computer program product of claim 19, wherein said determining the processing time comprises determining a lowest common denominator of periods defined by the plurality of corresponding frequencies, wherein the processing time is determined based on the lowest common denominator.
